(19) United States
(12) Patent Application Publication
(10) Pub. No.: US 2003/0134120 A1
(43) Pub. Date: Jul. 17, 2003

(54) Natural fiber coated with chitosan and a method for producing the same

(75) Inventors: Young-Jun Kim, Los Angeles, CA (US); Tae-Won Son, Daegu (KR); Won-Ki Kim, Daegu (KR); Hyun-Oh Yoo, Seoul (KR)

Correspondence Address: Nathan J . Prepelka, 700 Koppers Building, 436 Seventh Avenue, Pittsburgh, PA, 15219-1818, US

(73) Assignee: IBEKS TECHNOLOGIES CO., LTD.

(21) Appl. No.: 10/193,889

(22) Filed: Jul. 12, 2002

(30) Foreign Application Priority Data
Dec. 24, 2001 (KR) 2001-84171

(51) Int. Cl. B32B 27/12; B32B 27/04; A61K 9/70; D02G 3/00

(52) U.S. Cl. 428/375; 442/123; 8/181; 424/443

(57) ABSTRACT

Disclosed is a chitosan-coated natural fiber, comprising 70-99.9% by weight of a core consisting of a natural fiber; and 0.1-30% by weight of a sheath layer consisting of chitosan, uniformly coated over the surface of the natural fiber core. The chitosan-coated natural fiber is prepared by pretreating a natural yarn to improve affinity for chitosan, coating the pretreated natural fiber with chitosan, and stabilizing the fiber by heating or with an alkaline solution. The chitosan-coated fiber is 5-10 μm in fineness and 1-300 mm in length and shows desirable fiber properties as well as beneficial functions of chitosan, including antibacterial, deodorization and hemostatic activities.

### BACKGROUND OF THE INVENTION

1. Field of the Invention

The present invention relates, in general, to a natural fiber containing chitosan and, more particularly, to a natural fiber uniformly coated with chitosan, which can be used as a raw material not only for clothing with medical functions including antibacterial activity, hemostatic activity and tissue culture, but also for clothes, sheets and paper with antibacterial and deodorization activities. Also, the present invention is concerned with a method for producing such a natural fiber.

2. Description of the Prior Art

The term “natural fibers” as used herein comprises cellulose fibers such as cotton and linen, regenerated fibers such as rayon and acetate, and protein fibers such as wool and silk, as well as regenerated protein fibers such as meat protein fibers and casein fibers.

Useful as a raw material for clothing, paper, wooden products, etc., cellulose such as cotton, linen and pulp, which naturally occur in plants, are closely related to human daily life and are expected to find new and continuing various applications in the future, as before.

Protein fibers from animals have been used by mankind for 5,000 years. Particularly, wools, silks, angora wools and cashmeres have been widely used in human life. In the past, only natural protein fibers obtained from nature, such as fur, feather, wool, etc., were used. In recent times, there is remarkable demand for protein fibers. To meet the demand, protein fibers are prepared from various sources including animal flesh, milk, grains and beans.

Chitin is quantitatively found in the shells of crustaceans, such as crabs and shrimps, and insects, and in the cell walls of fungi, mushrooms and bacteria, and along with potassium carbonate, proteins, lipids, and pigments, serves to support the main structure of the shells and exoskeletons of various animals. Despite its abundance in nature, chitin has not been effectively utilized because of its low solubility in aqueous solutions. Owing to this problem, chitin is difficult to form into fibers or films and thus, has found limited applications.

U.S. Pat. No. 3,533,940 discloses a method for preparing chitosan from chitin, along with its application to fibers and films. For possible applications, the prepared chitosan is dissolved in aqueous organic solutions.

U.S. Pat. No. 4,699,135 teaches that chitin can be dissolved in polar solvents such as lithium chloride-containing dimethyl acetyl amide to produce chitin fibers. Also, disclosed is the production of chitosan staples from a solution of chitosan in an aqueous acetic acid solution.

U.S. Pat. No. 5,897,821 offers a method for manufacturing chitosan fiber wherein chitosan is dissolved in an aqueous solution of sodium thiocyanate and the resulting spinning solution is subjected to a wet spinning process to manufacture a chitosan fiber.

U.S. Pat. No. 5,900,479 describes the production of films and fibers of water-insoluble chitin using an aqueous organic acid solution of chitosan.

In addition to these, many other techniques for utilizing chitin or chitosan as raw materials in producing films and fibers are disclosed. In addition, active research has been directed to the production of biocompatible, hygienic products of chitin or chitosan fibers suitable for use in clinical medicine fields and to their possible applications. As a result, various relevant techniques are developed and disclosed at present.

Meanwhile, cellulose and chitosan, both a kind of polysaccharides, are very similar in structure, so that there is high physical and chemical compatibility therebetween. Taking advantage of these properties, a complex is prepared from cellulose fibers and chitosan, or cotton fabrics are coated with chisotan to produce functional fabrics.

For example, Noguchi, et al. (Kobunshi Kagaku, 30, 320-326, 1973) produced a cellulose/chitin conjugate fiber from cellulose xanthate and chitin xanthate in an alkali solution. Hasegawa et al. (J. Appl. Polym. Sci., vol. 45, 1837-1879, 1992) suggested the production of a cellulose/chitosan blended film from a solution of cellulose and chitosan in trifluoroacetic acid.

Hirano (Biotechnol. Ann. Rev., vol. 2, 237-258, 1996) disclosed chitin fibers, cellulose/chitin conjugate fibers, and chitosan-coated synthetic fabrics, and their functionalities, including wettability, antibacterial and deodorization activities.

U.S. Pat. No. 5,114,788 discloses a fabric having water absorption property and a method of manufacturing the fabric, in which a base fabric is immersed in or coated with a synthetic resin containing chitosan.

U.S. Pat. No. 5,306,550 discloses a biodegradable composite prepared from a mixture containing an aqueous solution of an acid salt of chitosan, a thermoplastic resin, and cellulose fibers.

In addition, based on the physiological and hygienic functions of chitosan, many functional clothes, fabrics and fibers employ chitosan fibers, cellulose-chitosan blend fibers, cellulose fiber-chitosan conjugate, and fabrics coated with chitosan-containing resins.

As described above, there are many techniques concerning the application of chitosan to fibers. However, most of the conventional techniques only suggest products made of fibers whose surfaces are partially coated with chitosan, or their production methods. Nowhere are found conjugate fibers whole surface of which is completely and firmly coated with chitosan.

Therefore, it is an object of the present invention to provide a sheath-core structure of a chitosan-coated natural fiber, in which chitosan is uniformly and firmly coated over the surface of a natural fiber and which shows beneficial effects of chitosan, with maintaining the desirable properties of natural fibers.

It is another object of the present invention to provide a method for producing such a chitosan-coated natural fiber.

### SUMMARY OF THE INVENTION

In accordance with an aspect of the present invention, there is provided a chitosan-coated natural fiber, comprising: 70-99.9% by weight of a core consisting of a natural yarn; and 0.1-30% by weight of a sheath layer consisting of chitosan, said sheath layer being uniformly coated over the surface of the natural fiber core.

In accordance with another aspect of the present invention, there is provided a method for producing a chitosan-coated natural fiber, comprising the steps of: pretreating a natural fiber in a solution at 0-90° C. for 1 min to 10 days, followed by dehydrating and conditioning the natural fiber at room temperature, so as to improve the affinity of the natural fiber for chitosan, said solution being selected from the group consisting of an aqueous alkaline solution, an aqueous acidic solution, an aqueous salt solution, and mixtures thereof; preparing a solution of chitosan by adding chitosan into the solvent selected from the group consisting of an aqueous acidic solution, an aqueous inorganic salt solution, an organic solvent, and mixtures thereof and aging the solution of chitosan; uniformly coating or penetrating the chitosan solution onto or into the pretreated natural yarn; and stabilizing the chitosan-coated or penetrated fiber by thermal or alkali treatment.

### BRIEF DESCRIPTION OF THE DRAWINGS

FIG. 1 is a process flow diagram showing the production of a chitosan-coated cellulose fiber in accordance with an embodiment of the present invention.

FIG. 2 is a schematic cross-sectional view showing a sheath-core structure of a chitosan-coated cellulose fiber in accordance with the present invention.

FIG. 3 is an electron microphotograph showing the surface of the chitosan-coated cellulose fiber in accordance with the present invention.

FIG. 4 is an electron microphotograph showing a cross section of the chitosan-coated cellulose fiber in accordance with the present invention.

FIG. 5 is an electron microphotograph showing a chitosan sheath which remains after the cellulose fiber forming the core of the sheath-core structure has been removed.

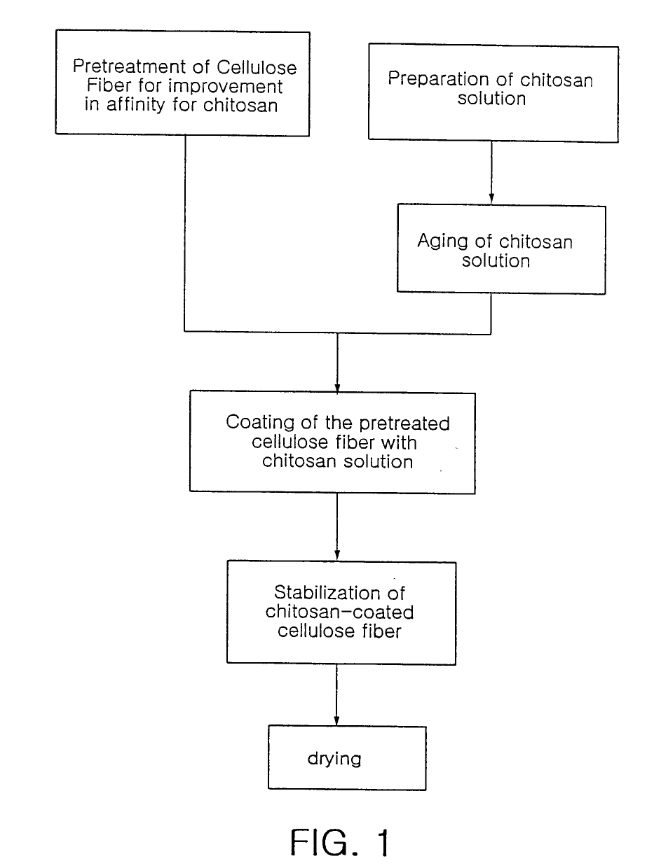

### DETAILED DESCRIPTION OF THE INVENTION

Based on the finding that chitosan is not only physically and chemically compatible with cellulose fiber, but also shows affinity for protein fibers due to the formation of hydrogen bond between the amide group of the fibroin structures in the protein fiber and the amine moiety of chitosan, the inventors conducted intensive and thorough research to apply chitosans to natural fibers and thereby to provide chitosan-coated natural fiber which shows both the beneficial effects of chitosan and desirable properties of the natural fibers.

According to the present invention, a chitosan solution is directly applied to the surface of a natural fiber without using an additive such as a binder or a coupling agent, to give a functional sheath-core fiber in which the natural fiber is firmly encapsulated by the chitosan.

Natural fibers, suitable for use in the present invention comprise cellulose fibers, prepared from seed, stem and leaf fibers, such as cotton, linen, sisal, abaca, kapok, flax, jute, ramie, hemp, kenaf; protein fibers including animal protein fibers such as wool, silk, cashmere, mohair, alpaca, and camel hair, meat protein fibers, casein fibers, and vegetable protein fibers; and regenerated fibers such as viscose rayon, copper ammonium rayon, polynosic rayon, lyocell, tencel, cellulose acetate, and cellulose triacetate.

Preferred are those fibers that contain water at an amount of 10% by weight or less and range from 4 to 80 μm in fineness and from 1 to 300 mm in length.

Suitable in the present invention is chitosan which ranges in polymerization degree from 20 to 10,000 and in deacetylation degree from 60 to 99.9%. More preferred is chitosan which ranges in polymerization degree from 100 to 5,000 and in deacetylation degree from 85 to 99%.

Chitosan is dissolved in an amount of 0.1 to 15% by weight in a solvent and preferably in an amount of 0.5 to 10% by weight. Any solvent may be used if it is selected from aqueous acidic solutions, aqueous inorganic salt solutions and organic solvents.

To obtain an aqueous acidic solution useful in the present invention, water is added with 0.1 to 20% by weight of an acid, examples of which include organic acids, such as acetic acid, lactic acid, formic acid, glycolic acid, acrylic acid, propionic acid, succinic acid, oxalic acid, ascorbic acid, gluconic acid, maleic acid, citric acid, glutamic acid, tartaric acid, toluene sulfonic acid, etc.; inorganic acids, such as hydrochloric acid, sulfuric acid, and phosphoric acid.

Available inorganic salt solutions contain an inorganic salt at an amount of 10-70 wt % in water. Examples of useful inorganic salts include sodium thiocyanate, zinc chloride, calcium chloride, sodium chloride, potassium chloride, and lithium chloride.

Useful organic solvents in the present invention are polar, examples of which include dimethylacetamide, N-methylpyrrolidone, dimethylformamide, diethylacetamide, trifluoroacetic acid, trichloroacetic acid, methylene chloride, and tetrachloroethane. In order to obtain higher polarity, one or more selected from the above-mentioned inorganic metal salts may be added at an amount of 1-10 wt % to the organic solvent.

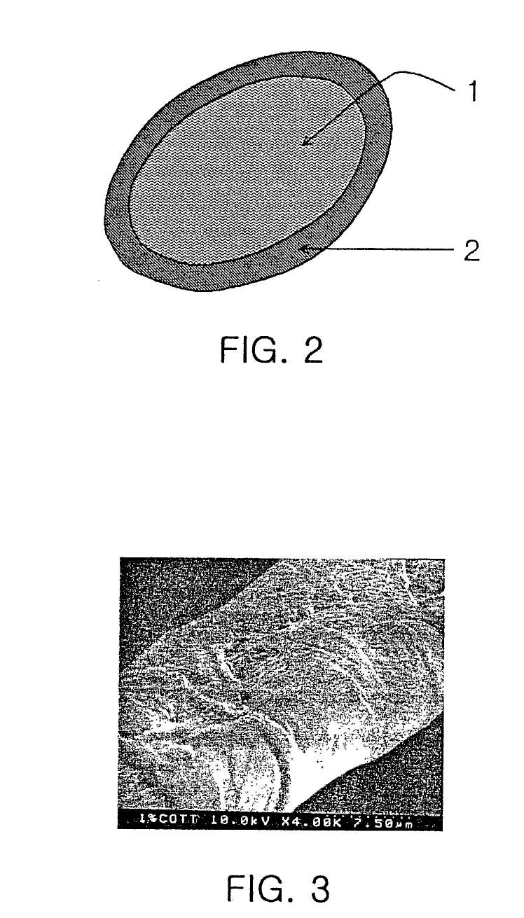

In the chitosan-coated natural fiber of the present invention, as schematically shown in FIG. 2, a natural fiber acts as a core 1 to which chitosan is uniformly and strongly attached to form a sheath layer 2.

The sheath-core fiber of the present invention 1-300 mm in length and 5-100 μm in fineness in which the inner core is responsible for a thickness of 4 to 80 μm, with the sheath layer in a thickness of 0.1 to 20 μm.

In the sheath-core fiber, the core fiber may be present in an amount of 70 to 99.9% by weight with the sheath layer amounting to 0.1 to 30% by weight. Besides, the sheath-core fiber of the present invention may comprise moisture and other components in an amount of 0.1 to 12% by weight.

When the content of the core fiber is below 70% by weight, chitosan is too abundant for the sheath-core conjugate to exhibit fiber properties. On the other hand, when the core fiber amount to more than 99.9% by weight, no chitosan coating effect is obtained.

Below, a description will be given of the production of the chitosan-coated natural fiber.

Conventionally, it was impossible to coat chitosan on the whole surface of a fiber. Even though chitosan is partially coated on the surface of a fiber, a binder or a coupling agent is additionally used to increase the lifetime of the chitosan coating, thereby ensuring its attachment onto the fiber.

By contrast, the present invention can provide a core-sheath structure with a great improvement in the adhesion of chitosan to the fiber, without using additional binders or coupling agents, through a series of processes including the pretreatment of the fiber, the aging of the chitosan solution, and post-coating fixation.

In order to increase the affinity between natural fibers and chitosan, natural fibers are subject to be pre-treated. This is achieved by immersing natural fibers in an aqueous alkali solution, an aqueous acidic solution, or an aqueous salt solution.

Useful in the present invention is an aqueous solution containing 1-40% by weight of an alkali metal oxide, examples of which include sodium hydroxide, potassium hydroxide, caldium hydroxide, lithium hydroxide, calcium oxide, barium hydroxide and barium oxide.

An aqueous acidic solution useful for the pretreatment of natural fibers is obtained by dissolving. in water 0.1 to 20% by weight of an organic acid which is exemplified by acetic acid, lactic acid, formic acid, glycolic acid, oxalic acid, succinic acid, propionic acid, acrylic acid, glycolic acid, tartaric acid, maleic acid, citric acid, and glutamic acid.

And inorganic salt solutions useful for the present invention contain an inorganic salt at an amount of 1-50 wt % in water. Examples of useful inorganic salts include organic metal salts, such as sodium acetate, sodium lactate, potassium acetate, potassium lactate, sodium glycolate, and potassium glycolate; alcohol metal salts such as sodium ethoxide; and inorganic metal salts such as sodium chloride, lithium chloride, calcium chloride, potassium chloride, zinc chloride, and sodium thiocyanate.

After being immersed in a suitable aqueous solution at 0-90° C. for 1 min to 10 days, the natural fibers are dehydrated and then conditioned at room temperature.

The pretreatment for improving the affinity of natural fiber for chitosan may be conducted once in an aqueous solution or twice or more in the same aqueous solution or different aqueous solutions.

With an improvement in affinity for and adhesion onto chitosan, the pretreated fibers allow chitosan to be coated uniformly over their surface.

In one embodiment of the present invention, as mentioned above, a chitosan solution uses as a solvent an aqueous acidic solution, an inorganic salt solution or an organic solvent. The chitosan solution contains chitosan in an amount of 0.1-15% by weight and more preferably 0.5-10% by weight.

To improve uniformity and adhesion onto fibers, the chitosan dissolved in the solvent is preferably aged at 0-50° C. for 1 hour to 10 days.

Next, the chitosan solution is applied to the fiber treated for affinity. The application may resort to any technique known in the art, including immersion and spraying. Preferably, the pretreated fiber is immersed in a chitosan solution.

As for the coating of fiber with a chitosan solution by an immersion technique, this is achieved by immersing the fiber in a chitosan solution to complete soak and removing excess chitosan solution. In a spraying technique, a chitosan solution is uniformly sprayed over the whole area of a fiber.

To fix the chitosan onto the fiber, a stabilization treatment is performed. This can be achieved by treating the chitosan-coated fiber thermally or with an aqueous alkali solution.

The stabilization by heat treatment is based on the fact that the chitosan coated on fibers is solidified by heating and thus prevented from separating from the fiber due to physical force or solvent solubilization. Preferably, the fiber is treated thermally at 60-160° C. for 1-100 min.

Based on neutralization with alkali, the stabilization by alkali treatment prevents the chitosan coated on the fiber from separating from the fiber owing to re-dissolution by external acid. This stabilization is performed by treating chitosan-coated fibers with an alkali solution at 0-80° C. for 1 min to 1 day.

Useful in the present invention is an aqueous solution containing 0.1-20% by weight of an alkali metal oxide, examples of which include sodium hydroxide, calcium hydroxide, potassium hydroxide, lithium hydroxide, calcium oxide, barium oxide and barium hydroxide.

To promote the stabilization by alkali treatment, an alcohol may be added in an amount of 0.1 to 10% by weight. Examples of useful alcohols include methanol, ethanol, propanol, butanol and phenol.

The alkali stabilization requires washing and drying processes after the alkali treatment.

With a sheath-core structure in which chitosan is firmly adhered to a cellulose core, forming a sheath layer, the stabilized, chitosan-coated fiber exhibits fiber properties and chitosan functions in concert. Thus, the conjugate fiber of the present invention is a novel substance in which natural fiber's biocompatibility coexists with chitosan functionalities.

Having generally described this invention, a further understanding can be obtained by reference to certain specific examples which are provided herein for purposes of illustration only and are not intended to be limiting unless otherwise specified.

### EXAMPLE 1

Treatment of Cellulose Fiber for Affinity

1 kg of cellulose fibers uniform in thickness was immersed in an aqueous 20 wt % sodium hydroxide solution to complete saturation, followed by compressing the fibers to remove excess sodium hydroxide solution. After being aged for 1 day at room temperature, the cellulose fibers were treated in an aqueous 3 wt % acetic acid solution at 20° C. for 3 hours and then dried.

Preparation of Chitosan Solution

0.05 kg of chitosan powder with a viscosity of 11.6 cps was dissolved in 0.95 kg of an aqueous 3 wt % lactic acid solution and the resulting solution was aged at 5-10° C. for 72 hours. This was added with 4 kg of water, and stirred completely to give a chitosan solution.

Production of Sheath-Core Conjugate Staple Fiber.

1 kg of the treated cellulose fibers was immersed in 5 liters of the chitosan solution to a completion and let to stand for 1 hour while being immersed. After the removal of excess chitosan solution by compression, the cellulose fibers were conditioned at room temperature for 3 hours. The conditioned, chitosan-coated cellulose fibers were, in part, dried at 60° C. for 60 min by use of a dryer. They were stabilized by thermal treatment at 150° C. for 10 min in a hot-air dryer to give a sheath-core structure.

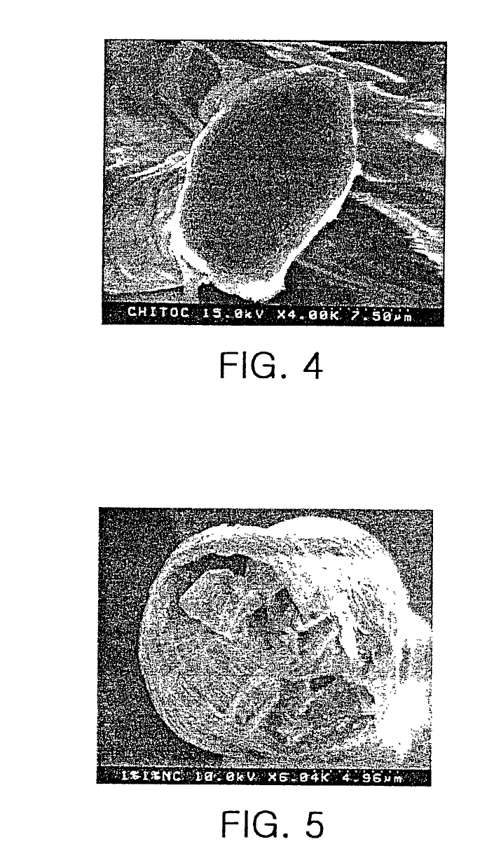

Using an electron microscope, the chitosan-coated fiber was examined for surface and cross-section conditions, and the results are given in FIGS. 3 and 4, respectively. As seen, chitosan were uniformly and firmly coated on the fiber.

Turning to FIG. 5, there is an electron microphotograph showing a chitosan sheath which remained after the cellulose fiber forming the core of the sheath-core structure was removed by use of dimethylsulfoxide (DMSO). As apparent from this figure, the chitosan was coated on the cellulose core, thickly and firmly.

### EXAMPLE 2

A chitosan-coated cellulose fiber was prepared in the same manner as in Example 1, with the modification that, instead of the stabilization through thermal treatment in a hot-air dryer, the stabilization of the fiber was achieved by chemical treatment in which the partially dried fiber was immersed in 5 liters of an aqueous 5 wt % sodium hydroxide solution at 20° C. for 1 hour and washed with hot and cool water to neutrality.

An examination showed that the inner core was completely covered with chitosan as in the fiber of Example 1, and the sheath layer was 0.5-4 μm in thickness.

### EXAMPELS 3 TO 5

Treatment with Different Solution for Affinity Improvement

1 kg of cellulose fibers ranging from 10 to 30 μm in fineness and from 40 to 80 mm in length was completely soaked in an aqueous 20 wt % sodium hydroxide solution, followed by compressing the fibers to remove excess sodium hydroxide solution. After being aged for 1 day at room temperature, the cellulose fibers were washed with water and then coated with chitosan (Example 3).

1 kg of cellulose fibers ranging from 10 to 30 μm in fineness and from 40 to 80 mm in length was treated in an aqueous 3 wt % acetic acid solution at 20° C. for 3 hours, dried and coated with chitosan (Example 4).

1 kg of cellulose fibers ranging from 10 to 30 μm in fineness and from 40 to 80 mm in length was treated with the aqueous sodium hydroxide solution and then with the aqueous acetic acid solution, followed by coating with chitosan (Example 5).

For use in coating the fiber, a chitosan solution was prepared by dissolving chitosan having a viscosity of 11.6 cps and a deacetylation degree of 99% to a concentration of 2% by weight in an aqueous 1 wt % lactic acid solution. This chitosan solution was coated on the treated cellulose fibers and excess chitosan solution present between cellulose fibers was removed, followed by stabilizing the fibers through treatment in an alkali solution. The chitosan-cellulose fibers in sheath-core structure were obtained after washing with water and drying at 60° C. for 3 hours.

The fibers were examined for their coating states and the results are given in Table 1, below.

TABLE 1

| Example No. | 3 | 4 | 5 |
|---|---|---|---|
| Treatment Sol. | Aq. NaOH | Aq. Acetic Acid | Aq. NaOH & |
| | | | aq. Acetic acid |
| Coated area | Whole surface | whole surface | whole surface |

### EXAMPLES 6 TO 12

Coating with Different Concentrations of Chitosan Solutions

Chitosan solutions were prepared by dissolving chitosan having a viscosity of 11.6 cps and a deacetylation degree of 99% to concentrations of 0.5, 1, 2, 3, 5, 7, and 9% by weight in aqueous 0.3, 0.5, 1, 1.5, 2.5, 3.5 and 4.5 wt % lactic acid solutions, respectively. These chitosan solutions were coated on the treated cellulose fibers and excess chitosan solutions present between cellulose fibers were removed, followed by stabilizing the fibers through treatment in an alkali solution. The chitosan-cellulose fibers in sheath-core structure were obtained after washing with water and drying at 60° C. for 3 hours.

The sheath-core structures were found to have more uniform thicknesses with the sheath layer adhering to the cores more firmly, as shown in Table 2, below.

TABLE 2

| Example No. | 6 | 7 | 8 | 9 | 10 | 11 | 12 |
|---|---|---|---|---|---|---|---|
| Chitosan Soln. Conc. (wt %) | 0.5 | 1 | 2 | 3 | 5 | 7 | 9 |
| Chitosan Sheath Thick. (μm) | 0.05-1 | 0.1-2 | 0.2-3 | 0.2-3 | 0.5-4 | 1-5 | 1-5 |

### EXAMPLES 13 TO 20

Solidification with Different Concentrations of Sodium Hydroxide Solution

A chitosan solution was prepared by dissolving chitosan having a viscosity of 11.6 cps and a deacetylation degree of 99% to a concentration of 2% by weight in an aqueous 0.5 wt % lactic acid solution. This chitosan solution was coated on the treated cellulose fibers and excess chitosan solution present between cellulose fibers was removed, followed by stabilizing the fibers through treatment in aqueous 0.5, 1, 2, 3, 4, 5, 7.5 and 10 wt % alkali solutions at 20° C. for 1 hour. The fibers were washed with hot and cold water to neutrality to stabilize the chitosan sheaths. Chitosan-cellulose fibers in sheath-core structure were obtained after washing with water and drying at 60° C. for 3 hours.

The fibers were examined for their coating states and the results are given in Table 3, below. Coat adhesion was measured by scraping the chitosan layers with fingernail.

TABLE 3

| Example No. | 13 | 14 | 15 | 16 | 17 | 18 | 19 | 20 |
|---|---|---|---|---|---|---|---|---|
| NaOH Conc. (wt %) | 0.5 | 1 | 2 | 3 | 4 | 5 | 7.5 | 10 |
| Adhesion | Excellent | Good | Good | Good | Good | Good | Excellent | Excellent |

Applied to spun yarn, non-woven fabrics and cottonwool, the chitosan-cellulose fiber of the present invention can be used as a raw material not only for clothing with medical functions including antibacterial activity, hemostatic activity and tissue culture, but also for clothes, sheets and paper with antibacterial and deodorization activities. The chitosan-cellulose fiber of the present invention can be spun in mixture with cotton, wool, polyester staple, nylon staple or acryl staple to give mixed-spun yarns. Also, the chitosan-cellulose fiber can be used for the production of functional non-woven fabrics.

Forming a sheath-core conjugate structure in which the cellulose fiber core is completely covered with the outer sheath chitosan, the chitosan-cellulose fiber of the present invention has the functional polymer chitosan over its whole surface and thus can be used as a novel substance which can exhibit as much chitosan functions even at a low amount as possible. Additionally, the sheath-core conjugate staple of the present invention, in which the cellulose fiber core is conjugated with the chitosan sheath, can be used as a multi-purpose material requiring a combination of the physical properties of cellulose fibers with the chemical functionalities of chitosan. For example, the sheath-core conjugate staple of the present invention can be used where functionality, processability, flexibility, toughness and duration are required.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A chitosan-coated natural fiber, comprising:
70-99.9% by weight of a core consisting of a natural fiber; and
0.1-30% by weight of a sheath layer consisting of chitosan, said sheath layer being uniformly coated over the surface of the natural fiber core.

2. The chitosan-coated natural fiber as set forth in claim 1, wherein the chitosan-coated natural fiber have from 5 to 100 μm of fineness and from 1 to 300 mm of length and the natural yarn is selected from the group consisting of a cellulose fiber, a protein fiber, a regenerated fiber, and mixtures thereof, said cellulose fiber being selected from the group consisting of cotton, linen, sisal, abaca, kapok, flax, jute, ramie, hemp, and kenaf, said protein fiber being selected from the group consisting of animal protein fiber, a meat protein fiber, a casein fiber, a vegetable protein fiber, and mixtures thereof, said animal protein fiber being selected from among wool, silk, cashmere, mohair, alpaca, and camel hair, said regenerated fiber being selected from the group consisting of viscose rayon, copper ammonium rayon, polynosic rayon, lyocell, tencel, cellulose acetate, cellulose triacetate, and mixtures thereof.

3. The chitosan-coated natural fiber as set forth in claim 2, wherein the natural yarn is a cellulose polymer having a polymerization degree of 100 to 20,000, with a water content of 10% by weight or less.

4. The chitosan-coated natural fiber as set forth in claim 1, wherein the chitosan ranges from 20 to 10,000 in polymerization degree and from 60 to 99.9% in deacetylation degree.

5. The chitosan-coated natural fiber as set forth in claim 4, wherein the chitosan ranges from 100 to 5,000 in polymerization degree and from 85 to 99% in deacetylation degree.

6. The chitosan-coated natural fiber as set forth in claim 1, wherein the core has from 4 to 80 μm of thickness and the sheath has from 0.1 to 20 μm of thickness.

7. A method for producing a chitosan-coated natural fiber, comprising the steps of:
pretreating a natural fiber in a solution at 0-90° C. for 1 min to 10 days, followed by dehydrating and conditioning the natural yarn at room temperature, so as to improve the affinity of the natural yarn for chitosan, said solution being selected from the group consisting of an aqueous alkaline solution, an aqueous acidic solution, an aqueous salt solution, and mixtures thereof;
preparing a chitosan solution by dissolving chitosan in a solvent, said solvent being selected from the group consisting of an aqueous acidic solution, an aqueous inorganic salt solution, an organic solvent, and mixtures thereof and aging the chitosan solution;
coating the chitosan solution to a desirable thickness uniformly over the pretreated natural fiber; and
stabilizing the chitosan-coated natural fiber by thermal or alkali treatment.

8. The method as set forth in claim 7, wherein the pretreatment step is carried out in an aqueous solution containing 1-40% by weight of an alkali metal oxide, said metal oxide being selected from the group consisting of sodium hydroxide, potassium hydroxide, caldium hydroxide, lithium hydroxide, calcium oxide, barium hydroxide, barium oxide, and mixture therof; in an aqueous acidic solution containing 0.1 to 20% by weight of an organic acid, said organic acid being selected from the group consisting of acetic acid, lactic acid, formic acid, glycolic acid, oxalic acid, succinic acid, propionic acid, acrylic acid, glycolic acid, tartaric acid, maleic acid, citric acid, glutamic acid, and mixtures thereof; or in an aqueous salt solution containing an organic salt, an alcohol metal salt or an inorganic salt in an amount of 1-50% by weight, said useful organic salt being selected from the group consisting of sodium acetate, sodium lactate, potassium acetate, potassium lactate, sodium glycolate, potassium glycolate, and mixtures thereof, said alcohol metal salt being sodium ethoxide, said inorganic salt being selected from the group consisting of sodium chloride, lithium chloride, calcium chloride, potassium chloride, zinc chloride, sodium thiocyanate, and mixtures thereof.

9. The method as set forth in claim 7, wherein the chitosan solution is prepared by dissolving chitosan in an aqueous acidic solution containing 0.1-20% by weight of an organic acid or an inorganic acid, said organic acid being selected from the group consisting of acetic acid, lactic acid, formic acid, glycolic acid, acrylic acid, malic acid, propionic acid, succinic acid, oxalic acid, ascorbic acid, gluconic acid, maleic acid, citric acid, glutamic acid, tartaric acid, toluene sulfonic acid, and mixtures thereof, said inorganic acid being selected from the group consisting of hydrochloric acid, sulfuric acid, and phosphoric acid; in an aqueous solution containing 10-70% by weight of an inorganic salt, said inorganic salt being selected from the group consisting of sodium thioisocyanate, zinc chloride, calcium chloride, sodium chloride, potassium chloride, lithium chloride, and mixtures thereof; or in an organic solvent selected from the group dimethylacetamide, N-methylpyrrolidone, dimethylformamide, diethylacetamide, trifluoroacetic acid, trichloroacetic acid, methylene chloride, tetrachloroethane, and mixtures thereof.

10. The method as set forth in claim 7, wherein the aging step is carried out at 0-50° C. for 1 hours to 10 days.

11. The method as set forth in claim 7, wherein the chitosan solution contains chitosan in an amount of 0.1-15% by weight.

12. The method as set forth in claim 11, wherein the chitosan solution contains chitosan in an amount of 0.5-10% by weight.

13. The method as set forth in claim 7, wherein the stabilization step is carried out by thermal treatment at 60-160° C. for 1-100 min.

14. The method as set forth in claim 7, wherein the stabilizing step is carried out using an alkaline solution at 0-80° C. for 1 min to 1 day.

15. The method as set forth in claim 14, wherein the alkaline solution is prepared by dissolving an alkali salt in an amount of 0.1-20% by weight in water, said alkali salt being selected from the group consisting of sodium hydroxide, calcium hydroxide, potassium hydroxide, lithium hydroxide, potassium oxide, barium oxide, barium hydroxide, and mixtures thereof.

16. The method as set forth in claim 14, wherein the alkaline solution is added with 0.1-10% by weight of an alcohol selected from the group consisting of methanol, ethanol, propanol, butanol, phenol, and mixtures thereof.
